(19) United States
(12) Patent Application Publication
(10) Pub. No.: US 2007/0015822 A1
(43) Pub. Date: Jan. 18, 2007

(54) Methods for selectively treating COX-2 mediated disorders by administering gamma-tocopherol

(76) Inventor: Peter Hanson, Suwanee, GA (US)

Correspondence Address: Judy Jarecki-Black, Ph.D,J.D, 3239 Satellite Blvd., Duluth, GA, 30096, US

(21) Appl. No.: 11/359,605

(22) Filed: Feb. 22, 2006

Related U.S. Application Data
(60) Provisional application No. 60/655,190, filed on Feb. 22, 2005.

(51) Int. Cl. A61K 31/355

(52) U.S. Cl. 514/458

(57) ABSTRACT

The present invention is based upon the novel observation of the COX-II-specific inhibitory activity of gamma-tocopherol and that by combining gamma-tocopherol with precursors of connective tissue constituents, injured or degenerated connective tissue, especially of articulated joints of animal patients, may be repaired. The method of the invention for treating a inflammatory disorder of a joint comprises administering to the mammal a pharmaceutical composition comprising an amount of gamma-tocopherol effective in selectively inhibiting cyclooxygenase-2 and at least one compound that elevates the production of a component of connective tissue in an amount effective for the promotion of connective tissue formation. The invention also provides pharmaceutical or veterinary compositions comprising a tocopherol preparation having at least 50% w/w gamma-tocopherol or a derivative thereof and in an amount effective for selectively inhibiting cyclooxygenase-2 in the recipient mammal and at least one compound that elevates the production of a component of connective tissue.

### RELATED APPLICATIONS/PATENTS & INCORPORATION BY REFERENCE

This application claims priority to provisional U.S. application Ser. No. 60/655,190 filed Feb. 22, 2005, the contents of which are hereby expressly incorporated herein by reference.

All documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention

### FIELD OF THE INVENTION

This invention provides for, inter alia, methods of treating cyclooxygenase-2 (“COX-2) mediated disorders by selectively inhibiting COX-2 in a mammal by administering a formulation comprising a COX-2 inhibiting amount of gamma-tocopherol. This invention also provides for compositions and methods for the treatment and repair of connective tissue and for the control of pain in a mammal suffering an inflammatory and degenerative connective tissue disorder by administering an effective amount of a formulation that comprises gamma-tocopherol and chondroitin and/or glucosamine or salts or derivatives thereof.

### BACKGROUND OF THE INVENTION

Inflammatory diseases such as asthma, hepatitis and rheumatoid arthritis, are significant causes of death or disability in humans and other mammals. Chronic inflammation contributes to the development of degenerative diseases including cardiovascular diseases, neuro-degenerative disorders and disorders of articulated joints. During inflammation, various eicosanoids derived from arachidonic acid (AA) play a key role in mediating the inflammatory response. For instance, prostaglandin E2 (PGE2) that results from cyclooxygenase (COX)-catalyzed oxidation of AA causes pain and fever, as well as activating cytokine formation. PGE2 can be produced by either the constitutive form (COX-1) or the inducible form (COX-2) of cyclooxygenase. In most inflammatory conditions, COX-2 is up-regulated and is the primary enzyme responsible for the formation of pro-inflammatory PGE2. Leukotriene B4 (LTB4), another oxidized product derived from AA through the 5-lipoxygenase-catalyzed pathway, is one of the most potent chemotactic agents. Because of the central roles of PGE2 and LTB4, COX-2 and 5-lipoxygenase have been recognized as key targets for drug therapy of inflammation-associated diseases. In particular, COX-2 inhibitors, which are classified as non-steriod anti-inflammatory drugs (NSAIDs) are effective in attenuating inflammatory response and offer effective therapies for certain inflammation-associated diseases.

Vitamin E includes of eight compounds; four tocopherols (alpha-, beta-, gamma-, and delta-) and four tocotrienols (alpha-, beta-, gamma-, and delta-). Among them, only alpha-tocopherol has been extensively studied. Gamma-tocopherol, however, is the major form of vitamin E in the US diet, but has drawn little attention compared with alpha-tocopherol, the primary form of vitamin E found in most dietary supplements. Delta-tocopherol is another form of vitamin E that is rich in some food sources (often found with gamma-tocopherol, e.g. in soybeans and soybean oil). Tocotrienols are mainly abundant in palm oil.

Vitamin E, and especially the tocopherols, are known in the art as antioxidants and nitrogen oxide scavengers, which are used to treat and prevent high blood pressure, thromboembolic disease, cardiovascular disease, cancer, natriuretic disease, the formation of neuropathological lesions, reduced immune system response, etc. or in foodstuffs for reducing nucleic acid damage in companion animals. See, e.g., U.S. Pat. Nos. 6,048,891; 6,242,749; 6,410,589; 6,242,362; US 2003/0022818 A1; 2003/0035821 and 2004/0102421.

It is known that gamma-tocopherol may be used as a COX-2 inhibitor for use in the treatment of inflammation. See, e.g., Q. Jaing et al., FASEB Journal (May 2003), 17, 816-22; S. Christian et al., J. Lipid Res. (2002), 43, 1978-85; Q. Jaing et al., Am. J. Clin. Nut. (2001), 74, 714-22; Q. Jaing et al., Free Radical Biology & Med., (2001), 31, S47; Q. Jaing et al., Proc. Natl. Assoc. Sci. U.S.A. (2000), 97, 11494-11499, K. O'Leary et al., Mutat. Res. (2004), 551, 245-254; and Q. Jaing, Proc. Natl. Assoc. Sci. U.S.A. (2000), 97, 11494-11499. US 2004/0102421 A1 teaches the use of anti-inflammatory compositions comprising phytylsubstituted chromanol and a NSAID cyclooxygenase inhibitor and indicates that gamma-tocopherol can be used to treat or prevent inflammatory disease because of its ability to block the PGE₂, LTB₄, and TNG-α pathways.

In addition to treating the inflammation that is associated with degenerative diseases of articulated joints such as rheumatoid arthritis or osteoarthritis, the art recognizes other agents, including such as chondroitin and its salts or glucosamine, that promote the repair of the connective tissue, cartilage, bone and joint lubricating fluids and not merely treat the symptoms. See, e.g., U.S. Pat. No. 5,364,845 or U.S. Pat. No. 5,916,565.

The connective tissues of mammals are constantly subjected to stresses and strains from mechanical forces that can result in painful or debilitating afflictions, such as arthritis, joint inflammation and stiffness. Such afflictions are especially acute in articulated joints, such as the neck, back, arms, hips, ankles and feet. However, the treatment of connective tissue afflictions can be problematic since an interruption in the trauma applied to the affected tissue often may not be possible, especially in the case of athletes and mammals such as race horses. Consequently, in those situations, treatment is usually directed to controlling the symptoms of the afflictions and not their immediate causes, regardless of the stage of the degenerative process.

Presently, steroids, such as corticosteroids, or other anti-inflammatory materials, such as NSAIDS, like high doses of aspirin, are widely used for the treatment of these ailments. See, for example, Vidal et al Pharmocol. Res. Commun. 10 557-569 (1978). In addition, hyaluronic acid and polysulfated glycosaminoglycan is also used in veterinary medicine, especially for treating equines. While these materials, however, often relieve pain and swelling associated with connective tissue disorders, almost all of the drugs currently available become progressively less effective. Furthermore, the drugs may also inhibit the body's own natural healing processes, exacerbating the deterioration of the damaged connective tissue.

The connective tissues repair themselves by manufacturing and remodeling prodigious amounts of collagen, the chief component of connective tissues, and the other major component of connective tissues, proteoglycans. This continual process is placed under stress when an injury occurs to connective tissues. In such cases, the production of connective tissue can double or triple compared to the normal rates, thereby increasing the demand for the constituent building blocks of both collagens and proteoglycans.

In the production of collagen, the rate-limiting step is maturation, rather than the production, of newly synthesized collagen. Excess collagen is simply degraded back to amino acids. Proteoglycans (PG's), however, have a specific rate-limiting reaction in their production, namely the conversion of glucose to glucosamine for the production of glycosaminoglycans (GAG's), a principal constituent of PG's.

Glucosamine is the key precursor to all of the modified sugars found in GAG's, including such as glucosamine sulfate, galactosamine and N-acetylglucosamine. Glucosamine also constitutes up 50% of hyaluronic acid which is the backbone of PG's on which other GAG's, like the chondroitin sulfates, are added. Once glucosamine is formed, the synthesis of GAG polymers and the synthesis of collagen inevitably follow.

Several disclosures have suggested bypassing the rate-limiting step of the conversion of glucose to glucosamine by providing exogenous glucosamine. For example, the intravenous administration of glucosamine, or derivatives thereof, has been disclosed in U.S. Pat. No. 3,232,836 issued to Carlozzi et al, for assisting in the healing of wounds on the surface of the body. In U.S. Pat. No. 3,682,076 issued to Rovati, the use of glucosamine and salts thereof are disclosed for the treatment of arthritic conditions. Finally, the use of glucosamine salts has also been disclosed for the treatment of inflammatory diseases of the gastrointestinal tract in U.S. Pat. No. 4,006,224 issued to Prudden. It has also been suggested to bypass the rate-limiting step by providing excess quantities of the modified sugars found in the GAG's. For example, in U.S. Pat. No. 3,6797,652 issued to Rovati et al, the use of N-acetylglucosamine is disclosed for treating degenerative afflictions of the joints.

Alternatively, excess quantities of the GAG's themselves (with and without various of the modified sugars) may be used. For example, in U.S. Pat. No. 3,371,012 issued to Furuhashi, a preservative is disclosed for eye graft material that includes galactose, N-acetylglucosamine (a modified sugar found in the GAG's) and chondroitin sulfate (a GAG). Additionally, U.S. Pat. No. 4,486,416 issued to Soll et al, discloses a method of protecting corneal endothelial cells exposed to the trauma of intraocular lens implantation surgery by administering a prophylactically effective amount of chondroitin sulfate. U.S. Pat. No. 5,141,928 issued to Goldman discloses the prevention and treatment of eye injuries using glycosaminoglycan polysulfates. These methods include the application of a corneal motor composition of fibronectin, chondroitin sulfate and collagen to the incision. In U.S. Pat. No. 4,801,619 issued to Lindblad, the intraarticular administration of hyaluronic acid is disclosed for the treatment of progressive cartilage degeneration caused by proteoglycan degradation.

What is needed are compositions, and methods for the use thereof, that combine the analgesic properties of an anti-inflammatory agent with compounds that can promote the repair of connective tissue damage, thereby improving the mobility of damaged articulated joints as well as relieving the pain and discomfort due to the injury.

Citation or identification of any document in this application is not an admission that such document is available as prior art to the present invention.

### SUMMARY OF THE INVENTION

The present invention addresses the need for compositions and methods for the use thereof that combine the analgesic properties of an anti-inflammatory agent with compounds that can promote the repair of connective tissue damage, thereby improving the mobility of damaged articulated joints. The present invention is based upon the novel and unexpected observation of the COX-II-specific inhibitory activity of gamma-tocopherol and that by combining gamma-tocopherol with precursors of connective tissue constituents, injured or degenerated connective tissue, especially of articulated joints of animal patients, may be repaired while the patient experiences the benefit of lessened pain and discomfort.

While the compositions and methods of the invention are contemplated to be suitable for treating a variety of inflammatory disorders, the invention is particularly useful for treating inflammatory disorders of connective tissues, especially of the cartilaginous and collagenous tissues of articulated joints. This invention, therefore, provides methods for treating a cyclooxygenase-2-mediated disorder in a mammal by selectively inhibiting cyclooxygenase-2, the method comprising administering to the mammal a pharmaceutical composition comprising an amount of gamma-tocopherol or a derivative thereof that is effective in selectively inhibiting cyclooxygenase-2 in the mammal.

The invention further provides a method for treating a inflammation or an inflammatory disease state in a mammal by selectively inhibiting cyclooxygenase-2, the method comprising administering to the mammal a pharmaceutical composition comprising a tocopherol preparation having at least 50% w/w gamma-tocopherol or an effective derivative or salt thereof and in an amount effective for selectively inhibiting cyclooxygenase-2 in the recipient mammal, optionally at least one compound that elevates the production of a component of connective tissue selected from chondroitin or glucosamine or a salt or derivative thereof or any combination thereof, and in an amount effective for the promotion of connective tissue formation, and optionally at least one pharmaceutically acceptable component selected from a pharmaceutical or veterinary excipient, additive or solvent.

In the various embodiments of this invention the concentration of gamma-tocopherol may be selected from, but not limited to, at least 50% w/w, at least 55% w/w, at least 60% w/w, at least 65% w/w, at least 70% w/w, at least 75% w/w, at least 80% w/w, at least 85% w/w, at least 90% w/w. In one advantageous embodiment of this aspect of the invention, at least 50% W/w of the tocopherol preparation is gamma-tocopherol. In one embodiment of this aspect of the invention, at least 60% w/w of the tocopherol preparation is gamma-tocopherol. In another embodiment, at least 75% w/wof the tocopherol preparation is gamma-tocopherol. In still another embodiment, at least 90% w/w of the tocopherol preparation is gamma-tocopherol.

In the various embodiments of this aspect of the invention, the subject the mammal can be companion animal and the inflammatory disease state can be rheumatoid arthritis or osteoarthritis.

In the embodiments of this aspect of the invention, the pharmaceutical composition may further comprise at least one of chondroitin or glucosamine or a salt or derivative thereof, and in an amount effective for the promotion of connective tissue formation and inflammation in an articulated joint.

The invention also provides pharmaceutical or veterinary compositions useful in the above methods, the compositions comprising a tocopherol preparation having at least 50% w/w gamma-tocopherol or an effective derivative or salt thereof and in an amount effective for selectively inhibiting cyclooxygenase-2 in the recipient mammal at least one compound that elevates the production of a component of connective tissue selected from chondroitin or glucosamine or a salt or derivative thereof or any combination thereof, and in an amount effective for the promotion of connective tissue formation.

In one embodiment of this aspect of the invention, the pharmaceutical or veterinary composition at least 60% w/w of the tocopherol preparation is gamma-tocopherol.

In another embodiment of the invention, the pharmaceutical or veterinary composition at least 75% w/wof the tocopherol preparation is gamma-tocopherol.

In yet another embodiment, the pharmaceutical or veterinary composition at least 90% w/w of the tocopherol preparation is gamma-tocopherol.

In various embodiments of the invention, the pharmaceutical or veterinary additive can be selected from a colorant, an antioxidant and a pH modifier, an excipient or a solvent.

Yet another aspect of the invention is a method for the treatment and repair of connective tissue and for the control of pain in a mammal in need thereof, which comprises administering an effective amount of a pharmaceutical or veterinary composition comprising a tocopherol preparation comprising at least 50% w/w gamma-tocopherol, at least one connective tissue precursor component selected from the group consisting of glucosamine and chondroitin, or a salt or derivative thereof, and optionally at least one of a pharmaceutical or veterinary excipient, additive or solvent. Inflammatory disease states that can be treated by the compositions and methods of the invention include, but are not limited to, for example, rheumatoid arthritis or osteoarthritis. Areas for treatment include articulated joints.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWING

The following detailed description, given by way of examples, but not intended to limit the invention solely to the specific embodiments described, may best be understood in conjunction with the accompanying drawings, in which:

FIG. 1 compares the efficacy of formulations of the present invention with differing dosage levels of gamma-tocopherol, a placebo and a NSAID to improve the condition of test animals using the urate crystal model for dog lameness;

FIG. 2 compares the effect of different concentrations and dosage levels of gamma-tocopherol, a placebo and a NSAID upon urate-induced lameness in dogs;

FIG. 3 illustrates the increase in sustainable force at two dosage levels of administered gamma-tocopherol, a placebo and a NSAID in the urate crystal model for dog lameness; and

FIG. 4 illustrates the increase in sustainable force at different concentrations and dosage levels of gamma-tocopherol, a placebo and a NSAID in the urate crystal model for dog lameness.

### DESCRIPTION OF THE INVENTION

This invention provides for a method for treating a cyclooxygenase-2 mediated disorder in a mammal by selectively inhibiting cyclooxygenase-2, the method comprising administering to the mammal a pharmaceutical composition comprising an amount of gamma-tocopherol or a derivative thereof that is effective in selectively inhibiting cyclooxygenase-2 in the mammal

The invention further provides a method for treating a inflammation or an inflammatory disease state in a mammal by selectively inhibiting cyclooxygenase-2, the method comprising administering to the mammal a pharmaceutical composition comprising a tocopherol preparation having at least 50% w/w gamma-tocopherol or an effective derivative or salt thereof and in an amount effective for selectively inhibiting cyclooxygenase-2 in the recipient mammal, optionally at least one compound that elevates the production of a component of connective tissue selected from chondroitin or glucosamine or a salt or derivative thereof or any combination thereof, and in an amount effective for the promotion of connective tissue formation, and optionally at least one pharmaceutically acceptable component selected from a pharmaceutical or veterinary excipient, additive or solvent.

The invention also provides pharmaceutical or veterinary compositions useful in the above methods, the compositions comprising a tocopherol preparation having at least 50% w/w gamma-tocopherol or an effective derivative or salt thereof and in an amount effective for selectively inhibiting cyclooxygenase-2 in the recipient mammal at least one compound that elevates the production of a component of connective tissue selected from chondroitin or glucosamine or a salt or derivative thereof or any combination thereof, and in an amount effective for the promotion of connective tissue formation.

While the compositions and methods of the invention are contemplated to be suitable for a treating a variety of inflammatory disorders, the invention is particularly useful for treating inflammatory disorders of connective tissues, especially of the cartilaginous and collagenous tissues of articulated joints, and most especially where there has been mechanical injury to the connective tissues of such a articulated joint.

Following longstanding law convention, the terms “a” and “an” as used herein, including the claims, are understood to mean “one” or “more”.

The term “cycloogenase-2” (COX-2) as used herein refers to the enzyme prostaglandin-endoperoxide synthase 2 (E.C. 1.14.99.1).

The term “mammal” as used herein refers to human and any non-human animals. The mammals may be domesticated companion animals such as, but not limited to, dogs, cats, rabbits, guinea pigs or livestock animals such as cattle, sheep, goats, horses, llamas and the like, or non-domesticated mammals found in wild environments or in captivity.

By “tocopherol” is meant any of a family of molecules (including both tocopherols and tocotrienols and derivatives thereof) which are characterized by a 6-chromanol ring structure and a side chain at the 2 position. Tocopherols possess a 4′,8′,12′-trimethyltridecyl phytol side chain, and the tocotrienols differ by the presence of double bonds at the 3′, 740 and 11′ positions of the side chain. As used herein, the term “tocopherol” refers to gamma-tocopherol, 3,4-dihydro-2,7,8-trimethyl-2-(4,8,12-trimethyltridecyl)-2H-1 -benzyopyran-6-ol; 2,7,8-trimethyl-2-(4,8,12-trimethyltridecyl)-6-chromanol; 7,8-dimethyltocol; o-xylotocopherol. As is known in the art, tocopherols and their derivatives can vary by the number and position of alkyl groups, double bonds and other substituents and variations on the ring and side chain. An “alkyl” is a cyclic, branched or straight chain chemical group containing only carbon and hydrogen, such as methyl, butyl and octyl. Alkyl groups can be either unsubstituted or substituted with one or more substituents, e.g., halogen, alkoxy, acyloxy, amino, hydroxyl, mercapto, carboxy, or benzyl. Alkyl groups can be saturated or unsaturated at one or several positions. Typically alkyl groups will comprise 1 to 8 carbons, preferably 1 to 6, and more preferably 1 to 4 carbon atoms. Additional tocopherols can be constructed by conjugation to the ring structure or side chain of various other moieties, such as those containing oxygen, nitrogen, sulfur and/or phosphorus. Tocopherol derivatives can also be made, as known in the art, by modifying the length of the side chain from that found in gamma-tocopherol. Tocopherols, including gamma-tocopherol, can also vary in stereochemistry and saturation of bonds in the ring structure and side chain.

Additional tocopherol derivatives, including prodrugs, can be made by conjugation of sugars or other moieties to the side chain or ring structure; these can serve any of a number of functions, including increasing solubility and increasing functional activity of the tocopherol. Thus, as is understood in the art, the invention encompasses the use of gamma-tocopherol derivatives in which substitutions, additions and other alterations have been made in the 6-chromanol ring and/or side chain, with the proviso that the derivatives maintain at least the functional activity of specific inhibition of COX-2 in animals. A “gamma-tocopherol” for use in the present invention can alternatively be a mixture of gamma-tocopherol derivatives. These mixtures include without limitation mixtures of stereoisomers of a single tocopherol (e.g., + and − stereoisomers of gamma-tocopherol; (±) indicates a racemic mixture) or mixtures of structurally distinct gamma-tocopherols. By “gamma-tocopherol derivative” is meant, therefore, gamma-tocopherol metabolites and synthetic chroman derivatives including, but not limited to, LLU-α, LLU-gamma, racemic chromans, chroman methyl esters, chroman esters, chroman amides, R₄ chroman esters, oxidized chroman derivatives, racemic 2,5,7,8-tetramethyl-2-(β-carboxyethyl)-6-hydroxy chroman, 2,5,7,8-tetramethyl-2-(β-carboxyethyl)-chroman, 2,7,8-trimethyl-2-(β-carboxyethyl)chroman, racemic 4-methyl-6-(5,6-dimethylbenzohinoyl)-4-hexanolid, 4-Methyl-6-(3,5,6-trimethylbenzochinoyl)-4-hexanolid, (S)-4-Methyl-6-(5,6-dimethylbenzochinoyl)-4-hexanolid, 2,7,8-Trimethyl-2-(β-carboxyethyl)-6-acetyl chroman, 2,7,8-Trimethyl-2-(β-carboxyethyl)-6-acetyl chroman methyl ester, and benzodipyran methyl ester. Other gamma-tocopherol metabolites and synthetic chroman derivatives may be known by those of skill in the art or will be discovered in the future and are encompassed by this definition.

The terms “inflammation” and “inflammatory disease” as used herein refer to the COX-2-mediated reaction of vascularized living tissue to injury. As such, inflammation is a fundamental stereotyped complex of cytologic and chemical reactions of affected blood vessels and adjacent tissues in response to an injury or abnormal stimulation caused by a physical, chemical or biological agent. Inflammation usually leads to the accumulation of fluid and blood cells at the site of injury, and is usually a healing process. However, inflammation sometimes causes harm, usually through a dysfunction of the normal progress of inflammation. Inflammatory diseases are those pertaining to, characterized by, causing, resulting from, or becoming affected by inflammation. Examples of inflammatory diseases or disorders include, without limitation, asthma, lung inflammation, chronic granulomatous diseases such as tuberculosis, leprosy, sarcoidosis, and silicosis, nephritis, amyloidosis, ankylosing spondylitis, chronic bronchitis, scleroderma, lupus, polymyositis, inflammatory bowel disease, ulcers, Sjorgen's syndrome, Reiter's syndrome, psoriasis, pelvic inflammatory disease, orbital inflammatory disease, and thrombotic disease. Among this group of diseases is rheumatoid arthritis, which is a chronic inflammatory disease of the joints, characterized by infiltration of T lymphocytes into the synovial fluid and eventual destruction of the cartilage and bones in the affected joints. Several studies have suggested that the infiltrating T lymphocytes are activated and cause neighboring tissue destruction.

The terms “effective amount” or “therapeutically effective amount” is meant to describe an amount of a compound of the present invention that is effective in inhibiting COX-2, in the case of gamma-tocopherol, or elevating the rate of synthesis of connective tissue such as collagen, thereby producing the desired therapeutic, ameliorative, inhibitory or preventative effect. Determination of a therapeutically effective amount is well within the capability of those skilled in the art, especially in light of the detailed disclosure provided herein. For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in other animal patients.

The term “pharmaceutical composition” refers to a preparation of one or more of the active ingredients described herein with other chemical components such as physiologically or pharmaceutically suitable carriers and excipients. The purpose of a pharmaceutical composition is to facilitate administration of a compound to an organism.

The term “active ingredient” refers to the compounds (e.g., gamma-tocopherol) accountable for the biological effect.

The terms “physiologically acceptable” and “pharmaceutically acceptable” which may be interchangeably as used herein refer to a carrier, a diluent, an additive, a solvent, a colorant, the therapeutically active agent, or any other component of the therapeutic composition that does not cause significant irritation to an organism and does not abrogate the biological activity and properties of the administered compound(s).

The term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

The term “connective tissue” as used herein refers to those tissues of an animal that comprise a high degree of matrix solid or resilient support material such as collagen, including cartilage, yellow or elastic tissue and white or collagenous fibrous tissue such as ligaments and tendons. Such tissues are found at the sites of articulated joints such as a knee, elbow, finger and toe joints and between the vertebrae. Such tissue provides resilience and articulation, and prevents adjacent bones from contacting during movement relative to each other. The connective tissues of articulated joints are subject to injury due to mechanical stress or inflammatory degenerative disease that results in the breakdown in the integrity of the tissue, resistance of movement and pain. Connective tissue in the context of the invention does not include the blood, but may include other connective tissues not anatomically associated with or integral with articulated joints.

Human and animal articular cartilage is highly specialized tissue, composed of chondrocytes embedded in an extracellular matrix. The matrix contains fibrillar components consisting mainly of collagen proteins, and non-fibrillar components, made up of proteoglycans, hyaluronic acid and water. Proteoglycan subunits consist of glycosaminoglycans (chondroitin and keratin sulfates) surrounding a protein core. Cartilage metabolism involves processes of synthesis, repair and degradation, which are ongoing and mediated by chondrocytes. When the balance among these processes is upset as in osteoarthritis and rheumatoid arthritis, cartilage damage results. The breakdown of the cartilage matrix is believed to be due to locally produced IL-1 from inflammatory cells increasing catabolic activity in adjacent chondrocytes. Oral glucosamine stimulates the manufacture of substances necessary for proper joint function and stimulate joint repair. Orally administered glucosamine sulfate is selectively taken up by the articular cartilage and stimulates the manufacture of glycosaminoglycan, a key structural component of cartilage. It also promotes the incorporation of sulfur into cartilage.

Gamma-tocopherol is a water-insoluble, non swelling amphiphile, as are triglycerides and cholesterol. Thus, many of the processes involved in the absorption of lipids are also required for absorption of gamma-tocopherol such as emulsification, solubilization within mixed bile salt micelles, uptake by the small intestine, packaging within lipoprotein particles, and secretion into the circulation via the lymphatic system. Gamma-tocopherol is transferred to tissues in much the same manner as other lipids and spontaneous transfer and exchange of tocopherol between cell membranes has been documented. Since gamma-tocopherol is rapidly absorbed in the lipids of various tissues including the liver, its antioxidant and radical scavenger activities primarily occur in the lipid phase and only tangentially in the aqueous phase. LLU-α, on the other hand, is considerably more hydrophilic than gamma-tocopherol and acts as an antioxidant, a natriuretic compound, and radical scavenger in primarily the aqueous phase. Thus, the present inventor contemplates a method to treat and prevent disease which employs supplements comprising gamma-tocopherol with and without fortification with racemic LLU-α (S)-LLU-α, or other gamma-tocopherol derivative so as to selectively provide selective COX-2 inhibition agents to the lipid and aqueous phases of a recipient animal's body.

Sources for gamma-tocopherol as well as the other isomeric forms of tocopherol are well known in the art; see, e.g., “The Merck Index” 12 ed., p. 1620, Merck & Co., Whitehouse Station, N.Y. (1996); U.S. Pat. No. 6,426,362; U.S. Pat. No. 6,410,589; U.S. Pat. Nos. 6,262,279; and 5,462,865; 4,122,094. Vitamin E is a mixture of a- and gamma-tocopherol. Vitamin E supplements consist primarily of the alpha form, whereas many sources derived from plants contain largely the gamma-form. This invention contemplates the use of tocopherols wherein at least 50% w/w of the tocopherols are gamma-tocopherols. In another embodiment tocopherol mixtures wherein are contemplated at least 60% w/w of the tocopherols are gamma-tocopherols. Yet another embodiment is contemplated wherein at least 75% w/w of the tocopherols are gamma-tocopherols, with mixtures wherein at least 90% w/w of the tocopherols are gamma-tocopherols are especially advantageous. It is further contemplated that the compositions of the invention may comprise salts or other derivatives of gamma-tocopherol or any combination thereof that retains or has enhanced COX-2-specific inhibitory activity.

Glucosamine is a component of all human and animal tissue and is found in especially high concentrations in the cartilage. Chemically an aminomonosaccharide, glucosamine provides the building blocks for the O-linked and N-linked glycosaminoglycans comprising the matrix of the connective tissues in the body. Over 90% of the sulfate form is readily absorbed from the small intestine. Of the absorbed glucosamine, 25% will be excreted in the urine, 65% excreted as exhaled carbon dioxide, and 10% remaining in the tissues. Once it is taken up into the chondrocytes of cartilage, glucosamine is incorporated into proteoglycans.

Similarly, chondroitin, glycosamine, β-glucan and/or phytosterols, and isoflavones as well as the pharmaceutically or veterinary salts of these compounds are well know in the art and are available either through commercial sources or by modifying known synthetic methods. One of skill in the art would have “Chemical Abstracts” at his or her disposal in order to prepare a specific compound. β-glucan is known in the art to reduce cholesterol and to function as an immunopotentiator. Further, it might have uses in treating diabetes. Phytosterols are also know to reduce cholesterol and may have a role in immunomodulation and in the prevention of cancer. Isoflavones are used as a supplement in post-menopausal women for their estrogen-like effects and may reduce cholesterol.

The subject compositions may be administered to effect various forms of release, which include, without limitation, immediate release, extended release, controlled release, timed release, sustained release, delayed release, long acting, pulsatile delivery, etc., using well known procedures and techniques available to the ordinary skilled artisan. A description of representative sustained release materials can be found in the incorporated materials in Remington's Pharmaceutical Sciences.

Other pharmaceutical or veterinary additives that can be included in the inventive formulations include a colorant, sweetener, antioxidant, or pH modifier or combinations thereof. The inventive formulations can also contain pharmaceutically acceptable organic and aqueous solvents known to those in the art and necessary for the solution of the components of the compositions herein.

Opacifiers may be added to absorb and/or reflect certain light and/or energy of certain wavelengths and may thus enhance the stability of the formulations. Opacifiers include, for example, zinc oxide or titanium dioxide and may be present in amounts from about 0.5 to 2.5%. Titanium dioxide is especially advantageous. These compounds are well known to practitioners of this art.

Additionally, the inventive formulations may contain other inert ingredients such as antioxidants, preservatives, or pH stabilizers. These compounds are well known in the formulation art. Antioxidant such as an alpha tocopherol (to an amount that does not reduce the proportion of gamma-tocopherol to less than 50% w/w of the total tocopherol in the formulation), ascorbic acid, ascobyl palmitate, fumeric acid, malic acid, citric acid, sodium ascorbate, sodium metabisulfate, n-propyl gallate, BHA (butylated hydroxyanisole), BHT (butylated hydroxytoluene), monothioglycerol and the like, may be added to the present formulation. The antioxidants are generally added to the formulation in amounts of from about 0.01 to about 2.0%, based upon total weight of the formulation, with about 0.05 to about 1.0% being especially advantageous. Preservatives, such as the parabens (methylparaben and/or propylparaben), are suitably used in the formulations in amounts ranging from about 0.01 to about 2.0%, with about 0.05 to about 1.0% being especially advantageous. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, and the like. Advantageous ranges for these compounds include from about 0.01 to about 5%.

Colorants may be added to the inventive formulations. Colorants contemplated by the present invention are those commonly known in the art. Specific colorants include, for example, dyes, an aluminum lake, caramel, colorant based upon iron oxide or a mixture of any of the foregoing. Especially advantageous are organic dyes and titanium dioxide. Advantageous ranges include from about 0.1% to about 25%.

Compounds that acidify the formulation are also contemplated. Again, acidifying compounds and their use to lower the pH of a formulation are well known to a practitioner in the art. Examples of such acidifying stabilizers include, but are not limited to compounds selected from the group consisting of ascorbic acid, malic acid, isoascorbic acid, cysteine hydrochloride, cysteine dihydrochloride, citric acid fumaric acid, acetic acid, sorbic acid, glycine hydrochloride, arginine hydrochloride, succinic hydrochloride, succinic acid, tartaric acid, phosphoric acid, hydrochloric acid, glucono-delta-lactone, and the like. Chelating agents may include, but are not limited to, EDTA, diethanolamine and triethanolamine.

The inventive topical formulations may also contain penetration enhancers, such as dimethylacetamide, Transcutol®, DMSO or dimethyl isorbide, or chelating agents. Penetration enhancers are used in small amounts, amounts that are of such quantity that they will not dissolve both actives.

Suitable routes of administration may, for example, include, but are not limited to, oral, rectal, transmucosal, especially transnasal, intestinal or parenteral delivery, including intramuscular, subcutaneous and intramedullary injections as well as intrathecal, direct intraventricular, intravenous, intranasal, or intraocular injections. Alternately, one may administer the pharmaceutical composition in a local rather than systemic manner, for example, via injection of the pharmaceutical composition directly into a tissue region of a patient. Preferably, the pharmaceutical compositions of the present invention are designed for oral, intramuscluar administration, or by direct delivery to the site of injury such as an injured articulated joint, as is detailed hereinafter.

The subject medicament compositions may be administered in conjunction with a carrier, vehicle or excipient suitable for use in pharmaceutical compositions. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition. Such carriers are well known in the pharmaceutical art as are procedures for preparing pharmaceutical compositions.

Depending on the intended route of delivery, the compositions may be administered in one or more dosage form(s) including, without limitation, liquid, solution, suspension, emulsion, tablet, multi-layer tablet, bi-layer tablet, capsule, gelatin capsule, caplet, lozenge, chewable lozenge, bead, powder, granules, dispersible granules, cachets, douche, suppository, cream, topical, inhalant, aerosol inhalant, patch, particle inhalant, implant, depot implant, ingestible, injectable, or infusion. The dosage forms may include a variety of other ingredients, including binders, solvents, bulking agents, plasticizers, etc.

The pharmaceutical compositions described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents that increase the solubility of the active ingredients to allow for the preparation of highly concentrated solutions.

For injection, the active ingredients of the pharmaceutical composition may be formulated in aqueous solutions, preferably in physiologically compatible buffers such as Hank's solution, Ringer's solution, or physiological salt buffer, or oil or adjuvant based solutions. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a recipient animal. Various oral formulations suitable for use in preparing the compositions of the present invention are described in U.S. Patent application 2004/0037869, incorporated herein by reference in its entirety. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, trehalose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Toxicity and the therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the physician in view of the patient's condition. (See e.g., Fingl, et al., 1975, in “The Pharmacological Basis of Therapeutics”, Ch. 1 pl).

The amount of a composition to be administered will, of course, be dependent on the subject being treated, the severity of the affliction, the manner of administration, the judgment of the prescribing veterinarian, etc. Dosage amount and interval may be adjusted individually to provide levels of the active ingredient at a targeted injury sufficient to suppress pain and inflammation at the site of connective tissue injury, and to promote the regeneration of the tissue (minimal effective concentration-MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The dosage forms of the present invention involve the administration of an active therapeutic substance or multiple active therapeutic substances in a single dose during a 24 hour period of time or multiple doses during a 24 hour period of time. The doses may be uneven in that each dose is different from at least one other dose.

A wide variety of dosages may be used, depending on the application and empirical determination; typical dosages range from 1 mg to 1 gram, preferably at least 10 mg, more preferably at least 100 mg. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions.

The preparation of soft gelatin capsules comprising commercially available gamma-tocopherol in doses of 200 to 800 mg is understood by those of skill in the art, although the inventors contemplate other methods of delivering the compositions of the invention, including as liquid, tablet, powder forms, including chewable formulation such as taught in U.S Patent application 2004/0037869 incorporated herein by reference in its entirety. The gamma-tocopherol may be present as the free alcohol or the acetate or succinate ester. Particularly advantageous compositions include at least 50% w/w gamma-tocopherol. These formulations are only intended to guide one of skill in the art and formulations of gamma-tocopherol that would be effective for use in the disclosed methods may include as low as 50% w/w gamma-tocopherol or up to 100% w/w gamma-tocopherol, but desirably contain between about 50% w/w gamma-tocopherol to about 95% w/w gamma-tocopherol.

Liquid forms suitable for oral administration may include a suitable aqueous or non-aqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

The above described components are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

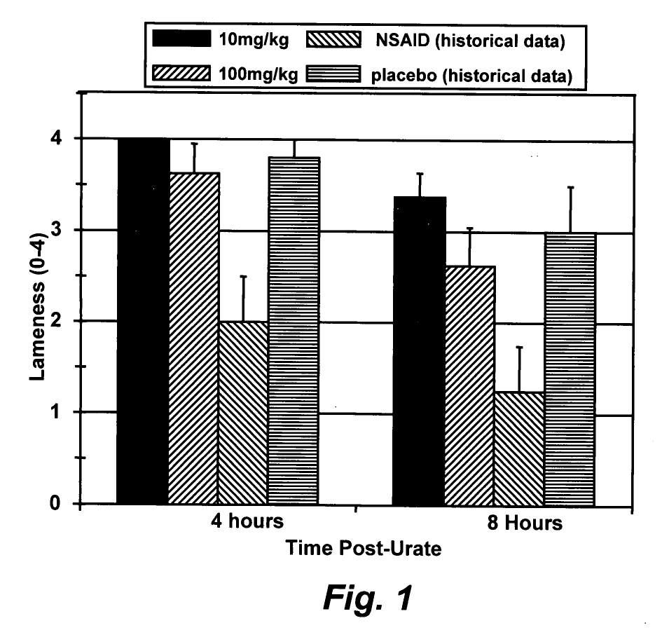

One aspect of the present invention, therefore, provides a method for treating a cyclooxygenase-2 mediated disorder in a mammal by selectively inhibiting cyclooxygenase-2 thereof, wherein the method comprises administering to the mammal a pharmaceutical composition comprising an amount of gamma-tocopherol or a derivative thereof that is effective in selectively inhibiting cyclooxygenase-2 in the mammal. The novel observation the gamma-tocopherol is a selective inhibitor of COX-2 in mammals is illustrated in Example 1, Table 1 below. The ability of gamma-tocopherol to relieve the pain induced by inflammation of an articulated joint of an animal is presented in Example 2 and FIGS. 1-4. In one embodiment of this aspect, the treated mammal is a companion animal.

Another aspect of the invention is a method for treating a inflammation or an inflammatory disease state in a mammal by selectively inhibiting cyclooxygenase-2, wherein the method comprising administering to the mammal a pharmaceutical composition comprising a tocopherol preparation having at least 50% w/w gamma-tocopherol or an effective derivative or salt thereof and in an amount effective for selectively inhibiting cyclooxygenase-2 in the recipient mammal, optionally at least one compound that elevates the production of a component of connective tissue selected from chondroitin or glucosamine or a salt or derivative thereof or any combination thereof, and in an amount effective for the promotion of connective tissue formation, and optionally at least one pharmaceutically acceptable component selected from a pharmaceutical or veterinary excipient, additive or solvent, wherein the gamma-tocopherol selectively inhibits inflammation and pain induced by cyclooxygenase-2 activity and optionally promotes the regeneration of connective tissue.

In the various embodiments of this invention the concentration of gamma-tocopherol may be selected from, but not limited to, at least 50% w/w, at least 55% w/w, at least 60% w/w, at least 65% w/w, at least 70% w/w, at least 75% w/w, at least 80% w/w, at least 85% w/w, at least 90% w/w. In one embodiment of this aspect of the invention, at least 60% w/w of the tocopherol preparation is gamma-tocopherol. In another embodiment, at least 75% w/w of the tocopherol preparation is gamma-tocopherol. In still another embodiment, at least 90% w/w of the tocopherol preparation is gamma-tocopherol.

In the various embodiments of this aspect of the invention, the mammal can be companion animal and the inflammatory disease state can be rheumatoid arthritis or osteoarthritis.

In the embodiments of this aspect of the invention, the pharmaceutical composition may further comprise at least one of chondroitin or glucosamine or a salt or derivative thereof, and in an amount effective for the promotion of connective tissue formation and inflammation in an articulated joint.

Another aspect of the invention is a pharmaceutical or veterinary composition comprising a tocopherol preparation having at least 50% w/w gamma-tocopherol or an effective derivative or salt thereof and in an amount effective for selectively inhibiting cyclooxygenase-2 in the recipient mammal, at least one compound that increases the production of a component of connective tissue selected from chondroitin or glucosamine or a salt or derivative thereof or any combination thereof, and in an amount effective for the promotion of connective tissue formation, and optionally at least one pharmaceutically acceptable component selected from a pharmaceutical or veterinary excipient, additive, wherein the pharmaceutical or veterinary additive is selected from a colorant, an antioxidant and a pH modifier or solvent.

In one embodiment of this aspect of the invention, the pharmaceutical or veterinary composition has at least 60% w/w of the tocopherol preparation as gamma-tocopherol.

In another embodiment of the invention, the pharmaceutical or veterinary composition has at least 75% w/w of the tocopherol preparation as gamma-tocopherol.

In yet another embodiment, the pharmaceutical or veterinary composition has at least 90% w/w of the tocopherol preparation as gamma-tocopherol.

In various embodiments of the invention, the pharmaceutical or veterinary additive can be selected from a colorant, an antioxidant and a pH modifier, the excipient can be trehalose and the solvent is ethanol or propylene glycol.

Yet another aspect of the invention is a method for the treatment and repair of connective tissue and for the control of pain in a mammal in need thereof, which comprises administering an effective amount of a pharmaceutical or veterinary composition comprising a tocopherol preparation comprising at least 50% w/w gamma-tocopherol, at least one connective tissue precursor component selected from the group consisting of glucosamine and chondroitin, or a salt or derivative thereof, and optionally at least one of a pharmaceutical or veterinary excipient, additive or solvent. Inflammatory disease states that can be treated by the compositions and methods of the invention include, but are not limited to, for example, rheumatoid arthritis or osteoarthritis. Areas for treatment include articulated joints.

It should be understood that the present invention is not limited to the specific compositions or methods described herein and that any composition having a formula or method steps equivalent to those described falls within the scope of the present invention. Preparation routes of the composition and method steps are merely exemplary so as to enable one of ordinary skill in the art to make the composition and use it according to the described process and its equivalents. It will also be understood that although the form of the invention shown and described herein constitutes advantageous embodiments of the invention, it is not intended to illustrate all possible forms of the invention. The words are words of description rather than of limitation. Various changes and variations may be made to the present invention without departing from spirit and scope of the invention.

The invention is illustrated by the following non-limiting examples:

### EXAMPLE 1

### Gamma-tocopherol is COX-2 Specific

A tocopherol composition comprising 66.6% gamma-tocopherol, or three known COX-2 inhibitors, at various concentrations was administered to six dogs and then the IC₅₀ was determined for each of the compounds. The results are summarized in Table 1 below.

TABLE 1

| Compound | COX-2 IC50-(μM) | COX-1 IC50-(μM) | COX-1:COX-2 |
|---|---|---|---|
| γ-Tocopherol | 27.8 | No activity | COX-2 |
| | | | selective |
| Carprofen | 10.0 | 68.6 | 7 |
| Deracoxib | 0.41 | 4.9 | 12 |
| Firocoxib | 0.31 | 119.1 | 384 |

IC50 COX-2 = 0.38 μM;
IC50 COX-1 = 27.2 μM
Gamma-tocopherol is selective for COX-2, having little or no detectable activity against COX-1.

### EXAMPLE 2

### Effectiveness of Treating Lameness Induced by Inflammation of Articulated Joints

A study was conducted to evaluate the effectiveness of the inventive formulations using the crystal urate-induced lameness model dogs. Formulations according to the invention and comprising 66.4% w/w or 90.9% w/w gamma-tocopherol and at 10 and 100 mg/kg body weight were administered to the dogs. The effects of gamma-tocopherol on the urate-induced lameness were scored after 4 hours or 8 hours as shown in FIGS. 1-4.

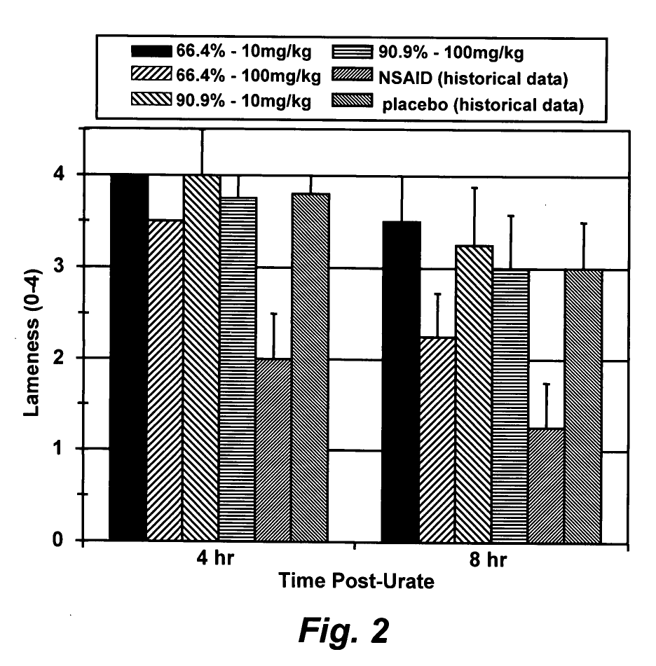

Improvement in the degree of lameness of the test animals was seen after 8 hours from administering 66.4% w/w or 90.9% w/w gamma-tocopherol, and at either of two dosage levels. Irrespective of the administered concentration, improvement was evident at both dosage levels, as shown in FIG. 1. A similar improvement in the condition of the test animals was seen at administered doses of 10 or 100 mg/kg body weight and with compositions containing either 66.4% w/w or 90.9% w/w gamma-tocopherol, as shown in FIG. 2

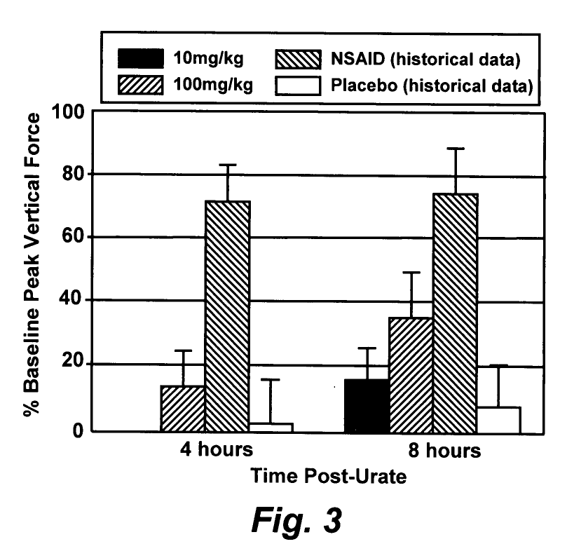

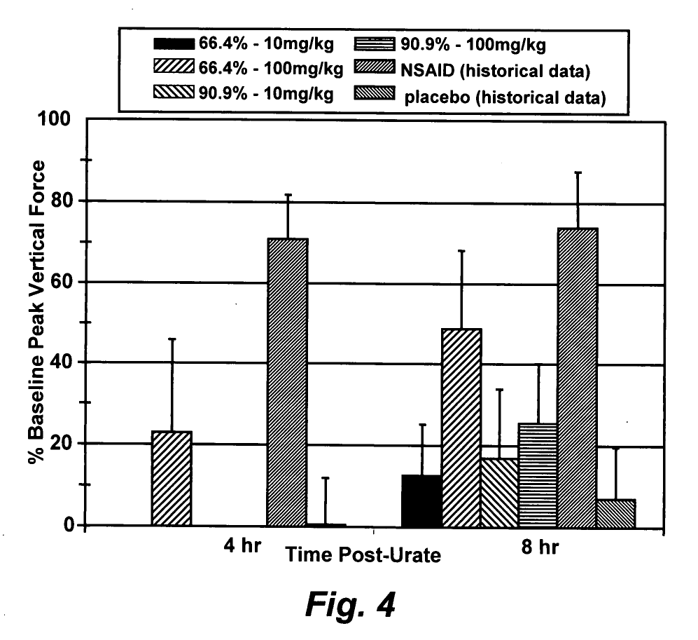

The degree of force upon the urate-treated joints tolerated by the test animals was increased after administration of gamma-tocopherol at either of the dose levels of 10 or 100 mg/kg body weight, as shown in FIG. 3 and at either concentration of 66.4% w/w or 90.9% w/w of administered gamma-tocopherol, as shown in FIG. 4.

What is claimed is:

1. A method for treating a cyclooxygenase-2 mediated disorder in a mammal by selectively inhibiting cyclooxygenase-2 thereof, wherein the method comprises administering to the mammal a pharmaceutical composition comprising a tocopherol preparation having at least 50% w/w gamma-tocopherol or an effective derivative or salt thereof and in an amount effective for selectively inhibiting cyclooxygenase-2 in the recipient mammal.

2. A method for treating a inflammation or an inflammatory disease state in a mammal by selectively inhibiting cyclooxygenase-2, wherein the method comprises administering to the mammal a pharmaceutical composition comprising (a) a tocopherol preparation having at least 50% w/w gamma-tocopherol or an effective derivative or salt thereof and in an amount effective for selectively inhibiting cyclooxygenase-2 in the recipient mammal, (b) at least one compound that increases the production of a component of connective tissue, said compound being selected from chondroitin or glucosamine or a salt or derivative thereof, or any combination thereof, and in an amount effective for the promotion of connective tissue formation, and (c) optionally at least one pharmaceutically acceptable component selected from a pharmaceutical or veterinary excipient, additive or solvent, wherein the pharmaceutical composition selectively inhibits inflammation and pain induced by cyclooxygenase-2 activity and promotes the regeneration of connective tissue.

3. The method according to claim 2, wherein the tocopherol preparation comprises at least 60% w/w gamma-tocopherol, at least 75% w/w gamma-tocopherol, or at least 90% w/w gamma-tocopherol.

4. The method according to claim 2, wherein the inflammatory disease state is rheumatoid arthritis or osteoarthritis.

5. The method according to claim 2, wherein the pharmaceutical composition is delivered to an articulated joint.

6. A pharmaceutical or veterinary composition comprising:
(a) a tocopherol preparation having at least 50% w/w gamma-tocopherol or an effective derivative or salt thereof and in an amount effective for selectively inhibiting cyclooxygenase-2 in the recipient mammal;
(b) at least one compound that increases the production of a component of connective tissue, said compound being selected from chondroitin or glucosamine or a salt or derivative thereof, or any combination thereof, and in an amount effective for the promotion of connective tissue formation; and
(c) optionally at least one pharmaceutically acceptable component selected from a pharmaceutical or veterinary excipient, additive, wherein the pharmaceutical or veterinary additive is selected from a colorant, an antioxidant and a pH modifier or solvent.

7. The pharmaceutical or veterinary composition according to claim 6, wherein the tocopherol preparation comprises at least 50% w/w, at least 55% w/w, at least 60% w/w, at least 65% w/w, at least 70% w/w, at least 75% w/w, at least 80% w/w, at least 85% w/w, or at least 90% w/w gamma-tocopherol.

8. The pharmaceutical or veterinary composition according to claim 6, wherein the composition is a component of a kit, said kit comprising packaging material, a vessel containing the pharmaceutical or veterinary composition, and instructions for the use of the composition for selectively inhibiting cyclooxygenase-2 in a recipient mammal and regenerating injured connective tissue thereof.
